(19) United States
(12) Patent Application Publication
(10) Pub. No.: US 2005/0191303 A1
(43) Pub. Date: Sep. 1, 2005

(54) Interleukin-18-binding protein

(75) Inventors: Kakuji Torigoe, Okayama (JP); Madoka Taniai, Okayama (JP); Masashi Kurimoto, Okayama (JP)

Correspondence Address: BROWDY AND NEIMARK, P.L.L.C., SUITE 300, 624 NINTH STREET, N.W., WASHINGTON, DC, 20001-5303, US

(73) Assignee: KABUSHIKI KAISHA HAYASHIBARA SEIBUTSU KAGAKU KENKYUJO, Okayama-shi (JP)

(21) Appl. No.: 10/876,572

(22) Filed: Jun. 28, 2004

Related U.S. Application Data
(62) Division of application No. 09/786,130, filed on Mar. 1, 2001, now abandoned.

(30) Foreign Application Priority Data
Sep. 1, 1998 (JP) 247588/1998
Nov. 18, 1998 (JP) 327914/1998

(51) Int. Cl. A61K 38/17; A61K 39/395

(52) U.S. Cl. 424/145.1; 514/12

(57) ABSTRACT

The objects of this invention are to provide a substance which suppresses the physiological activities of IL-18 through binding to IL-18, uses of the substance, and a DNA encoding the substance; this invention attains these objects by providing an IL-18-binding protein comprising a specific amino acid sequence, a DNA encoding this protein, and an IL-18-suppressor as well as agent for susceptive diseases containing as an effective ingredient this IL-18-binding protein.

### CROSS-REFERENCE TO RELATED APPLICATIONS

This is a divisional of copending parent application Ser. No. 09/786,130, filed Mar. 1, 2003, which is a national stage under 35 U.S.C. 371 of international application PCT/JP98/05186, filed Nov. 18, 1998, which designated the United States.

### TECHNICAL FIELD

This invention relates to a novel cytokine-binding protein, particularly, an interleukin-18-binding protein.

### BACKGROUND ART

Interleukin-18 (hereinafter abbreviated as “IL-18”) is a type of cytokine that transduces signals in immune system. As documented in Japanese Patent Kokai Nos. 27,189/96 and 193,098/96 and Haruki Okamura et al., “Nature,” Vol. 378, No. 6552, pp. 88-91 (1995), IL-18 was designated “interferon-γ inducing factor (IGIF)” immediately after its discovery; this designation was changed later into “IL-18 (interleukin-18)” in accordance with the proposal in Shimpei Ushio et al., “The Journal of Immunology,” Vol. 156, pp. 4274-4279 (1996). As described in “The Cytokine Handbook,” edited by Angus W. Thomson, published by Academic Press Ltd. (1998), pp. 465-489, mature IL-18 consists of 157 amino acids and has the activities of inducing the production of interferon-γ (hereinafter abbreviated as “IFN-γ”), which is useful as a physiologically active protein, by immunocompetent cells, as well as of enhancing the cytotoxicity of killer cells and inducing the generation of killer cells. Because of these activities, IL-18 has been deemed useful in various pharmaceuticals, for example, an anti-viral agent, anti-microbial agent, anti-tumor agent, and anti-immunopathic agent. Energetic studies are now in progress to realize these potential uses.

As mentioned above, IL-18, like other cytokines, is inherently produced and secreted as a substance responsible for signal transduction in immune system. Therefore, excessive amounts of IL-18 may disturb the balance of immune system when over-produced or excessively administered in the body of mammals. Recent studies have demonstrated that patients with autoimmune diseases including rheumatoid arthritis are significantly higher in IL-18 level in their body fluids than healthy humans, as disclosed in Japanese Patent Kokai No.96730/98. This indicates the possibility that IL-18 directly or indirectly relates to the crisis of certain diseases. In this field, as well as for the clarification in physiological activities and practical utilization of IL-18, there is a great demand for earlier clarification and utilization of a substance which suppresses the physiological activities of IL-18.

In view of the foregoing, the first object of this invention is to provide a substance which is capable of suppressing the physiological activities of IL-18 and applicable to humans and other mammals.

The second object of this invention is to provide a DNA encoding the substance.

The third object of this invention is to provide uses of the substance as an IL-18-suppressor.

The fourth object of this invention is to provide uses of the substance as a pharmaceutical.

### DISCLOSURE OF INVENTION

The present inventors energetically studied to attain the above objects. As a result of theses studies, the inventors found a substance in mammalian body fluids which suppresses the physiological activities of IL-18 through binding to IL-18. The inventors then isolated this substance and investigated for its characteristics and properties. This substance was proved in the nature of a protein, and exhibited the ability of binding to IL-18 and thus suppressing the physiological activities thereof even in the isolated form. Further, this IL-18-binding protein, thus identified, was found to have an efficacy in treatment and prevention of various diseases resulting from augmented immunoreactions such as autoimmune diseases, inflammatory diseases, and allergic diseases, when administered to humans and other mammals.

Specifically, this invention attains the first object by providing the IL-18-binding protein comprising a part or the whole of the amino acid sequence shown in SEQ ID NO:1 or 2.

This invention attains the second object by providing a DNA encoding this IL-18-binding protein.

This invention attains the third object by providing an IL-18-suppressor containing as an effective ingredient this IL-18-binding protein.

This invention attains the fourth object by providing an agent for susceptive diseases containing as an effective ingredient this IL-18-binding protein.

### BRIEF DESCRIPTION OF DRAWINGS

FIG. 1. shows peptide maps of the IL-18-binding protein of human origin. The chromatogram A is the peptide map obtained after trypsin digestion, and the chromatogram B is that obtained after trypsin-pepsin digestion. The numerals 1 to 20 indicate the eluted positions of the peptide fragments 1 to 20 which were analyzed for amino acid sequence.

FIG. 2. shows peptide maps of the IL-18-binding protein of mouse origin. The chromatogram A is the peptide map obtained after trypsin digestion, and the chromatogram B is that obtained after trypsin-pepsin digestion. The numerals 1 to 8 indicate the eluted positions of the peptide fragments 1 to 8 which were analyzed for amino acid sequence.

FIG. 3. shows a restriction enzyme map of a recombinant DNA comprising a nucleotide sequence encoding the IL-18-binding protein of human origin.

FIG. 4. shows a restriction enzyme map of a recombinant DNA comprising a nucleotide sequence encoding the IL-18-binding protein of mouse origin.

In the figures, the meanings of the symbols are as follows:
EFH18BPH6 cDNA, cDNA comprising a nucleotide sequence encoding the IL-18-binding protein of human origin;
EFM18BPH-MK2 cDNA, cDNA comprising a nucleotide sequence encoding the IL-18-binding protein of mouse origin;
EF1αP, elongation factor 1 promotor;
Amp, ampicillin-resistant gene; and
ori, replication origin.

### BEST MODE OF INVENTION

The following are to explain the best mode of this invention; the protein of this invention is characterized by the property of suppressing the physiological activities of IL-18 through binding to IL-18 and by its specific amino acid sequences. The IL-18-binding protein of this invention, when acting on IL-18, suppresses the representative physiological activity of IL-18, inducing IFN-γ production by immunocompetent cells. Further, the IL-18-binding protein of this invention, when binding to IL-18, may suppress the enhancement of cytotoxicity of killer cells and the induction of killer cell generation by the action of IL-18. The IL-18-binding protein of this invention comprises a part or the whole of the amino acid sequence shown in SEQ ID NO:1 or 2 in the sequence listing; for example, the IL-18-binding protein of human origin comprises as a partial amino acid sequence(s) a part or the whole of the amino acid sequence shown in at least one of SEQ ID NOs:3 to 23, and the IL-18 binding protein of mouse origin comprises as a partial amino acid sequence(s) a part or the whole of the amino acid sequences shown in at least one of SEQ ID NOs:24 to 31. In body fluids such as urine and blood, the IL-18-binding protein of this invention usually exists as a soluble protein, which exhibits, on SDS-polyacrylamide gel electrophoresis, a protein band bearing IL-18-binding ability at a molecular weight of about 40,000 to about 60,000 daltons.

The IL-18-binding protein of this invention can be obtained from mammalian body fluids and cells by studying them for the above characteristics as criteria. The body fluids include bloods, lymphs, ascites, and urines, and the cells include epidermal cells, endothelial cells, interstitial cells, chondrocytes, monocytes, lymphocytes, neurocytes, and cell lines establishable from these cells. With regard to cost for preparation, it is advantageous to apply recombinant DNA techniques with a DNA encoding the IL-18-binding protein of this invention. DNAs encoding the IL-18-binding protein of this invention can be obtained by screening mammalian genes on the basis of the amino acid sequences shown in SEQ ID NOs: 1 to 31. A DNA of human origin encoding the IL-18-binding protein of this invention usually comprises a part or the whole of the nucleotide sequence shown in SEQ ID NO:32, and a DNA of mouse origin usually comprises a part or the whole of the nucleotide sequence shown in SEQ ID NO:33. Mammalian or microbial host cells transformed with such DNAs can produce the IL-18-binding protein of this invention at relatively high yields, when the cells are cultured in a usual manner. The mammalian host cells include, for example, 3T3 cells (ATCCCCL-92), C127I cells (ATCCCRL-1616), CHO-K1 cells (ATCCCCL-61), CV-1 cells (ATCC CCL-70), COS-1 cells (ATCC CRL-1650), HeLa cells (ATCC CCL-2), MOP 8 cells (ATCC CRL-1709), mutant strains from these cells, and other epidermal cells, interstitial cells, and hemopoietic cells of human, monkey, mouse, or hamster origin. The microbial host cells include, for example, bacteria, fungi, and yeasts. Among these host cells, mammalian host cells and yeasts are more advantageous for the production of the IL-18-binding protein in the form of a glycoprotein.

To prepare the IL-18-binding protein of this invention from the sources as described above, the body fluids or the cellular or microbial cultures can be disrupted if necessary, for example, by sonication, and then subjected to conventional methods to purify physiologically active proteins. The conventional methods include salting-out, dialysis, filtration, concentrating, separatory sedimentation, ion-exchange chromatography, gel filtration chromatography, adsorption chromatography, isoelectric focusing chromatography, hydrophobic chromatography, reversed phase chromatography, affinity chromatography, gel electrophoresis, and isoelectric focusing electrophoresis, which can be applied alone or in combination.

Immune system inherently functions to protect a living body from foreign noxious substances, but under certain conditions, this function rather causes injurious affections to the living body. In the case of organ transplantation such as grafting skins, kidneys, livers, hearts, bone marrows to mammals, rejection reactions against alloantigens may activate T cells, induce lymphocyte proliferation, and then cause inflammation. While differently in symptoms, similar phenomena can be observed in the case of invasion of exogenous antigens such as allergens that a host recognizes as non-self. In autoimmune diseases, substances that should be recognized as self by a host induce allergic reactions.

Because the IL-18-binding protein of this invention functions as an agent to suppress the physiological activities of IL-18 through binding to IL-18, which is responsible for activation of immune system, the protein of this invention is expected to suppress immunoreactions as described above when administered to humans and other mammals. Therefore, the term “susceptive diseases” as referred to in this invention includes immunopathies resulting from augmented immunoreactions in general, such as rejection reactions and allergic reactions, and the diseases that can be treated or prevented by the direct or indirect action of the IL-18-binding protein of this invention. The susceptive diseases include, for example, the above-mentioned rejection reactions associated with organ transplantation, active chronic hepatitis, atrophic gastritis, autoimmune hemolytic anemia, Basedow's disease, Behcet's syndrome, CRST syndrome, cold agglutination hemolytic anemia, ulcerative colitis, Goodpasture's syndrome, hyperthyroidism, chronic thyroiditis, idiopathic thrombocytopenic purpura, juvenile diabetes, leukopenia, multiple sclerosis, severe myasthenia, paroxysmal cold hemoglobinuria, pernicious anemia, polyarteritis nodosa, multiple myositis, primary biliary cirrhosis, rheumatic fever, rheumatoid arthritis, Hashimoto's disease, Sjögren's syndrome, Crohn's disease, sympathetic ophthalmia, progressive systemic sclerosis, Wegener's granulomatosis, HIV infection, asthma, atopic dermatitis, allergic rhinitis, pollinosis, apitoxin allergy, and other autoimmune, inflammatory, and allergic diseases in general. The IL-18-binding protein of this invention has another efficacy to treat or prevent septic shock resulting from excessively produced or administered IFN-γ. In a living body, IL-18 possibly augments Fas-ligand production, and inversely, Fas-ligand possibly induces IL-18 secretion from cells. The IL-18-binding protein is therefore efficacious in treatment and prevention of immunopathies relating to Fas and to Fas-ligand in general. In addition, the IL-18-binding protein of this invention is efficacious in treatment or prevention of hepatic disorders such as viral hepatitis, alcoholic hepatitis, toxic hepatitis, fulminant hepatitis, viral cirrhosis, alcoholic cirrhosis, toxic cirrhosis, biliary cirrhosis, fatty liver, hepatic tumors, and hepatic angiopathies, cholesystopathies or biliary disorders such as cholangitis, cholecystitis, primary sclerosing cholangitis, cholecystic tumors, and biliary tumors, pancreatopathies such as acute pancreatitis, chronic pancreatitis, deficiency in pancreatic functions, pancreatic tumors, and hydrocyst, as well as in alleviation or improvement of symptoms associated with these disorders, for example, inappetence, malaise, fatigue, bellyache, dorsalgia, icterus, fever, hepaticencephalosis, ascites, hemorrhagic determination, and other dyshepatia and hepatargia. In these cases, a medicament(s) capable of activating hepatic functions such as protoporphyrin, thioprine, malotilate, liver hydrolyzates, glycyrrhizin, dichloroacetate diisopropylamine, methylmethionine sulfonium chloride, glutathione, taurine, cyanidanol, interferons, vitamin B1, vitamin B2, vitamin B6, vitamin B12, thioctic acid, hsiao-tz\check{u}-ku-t [symbol] ang, ta-tz\check{u}-ku-t [symbol] ang, tz\check{u}-ku-kuei-chih-t [symbol] ang, aspartic acid, glycyrrhiza, methionine, thioprine, and glycyrrhizin can be used in combination. The IL-18-binding protein further additionally has an efficacy to alleviate or prevent disorders in circulatory system such as ischemia, ischemic cardiomyopathy, cerebral ischemia, basilar artery migraine, abnormal vascularnet at the brain base, cerebral apoplexy, aneurysm at the brain base, arteriosclerosis, disorders in vascular endothelium, diabetes, mesenteric angiemphraxis, and superior mesenteric artery syndrome and disorders in nerve system such as Parkinson's disease, spinomuscular amyotrophy, amyotrophic sclerosis at the funiculus lateralis, Alzheimer's disease, dementia, cerebrovascular dementia, AIDS dementia, and encephalomyelitis. As above, the agent for susceptive diseases of this invention, containing the IL-18-biding protein as an effective ingredient, has a variety of uses to treat or prevent the above-mentioned susceptive diseases, for example, as an anti-autoimmune agent, anti-inflammatory agent, anti-allergic agent, anti-tumor agent, immunosuppressant, hemopoietic agent, thrombopoietic agent, lenitive agent, antipyretic agent, and agent to improve hepatic functions. The agent for susceptive diseases of this invention is usually prepared in the form of a liquid, suspension, paste, or solid, and contains the IL-18-binding protein of this invention in a content of 0.00001-100% (w/w), preferably, 0.0001-20% (w/w), while the content may vary depending on the form of this agent as well as the types and symptoms of the susceptive diseases to be treated.

The agent for susceptive diseases of this invention includes those in the form consisting of the IL-18-binding protein of this invention alone and in the form of a composition comprising this protein and one or more of other physiologically acceptable, for example, adjuvants, extenders, diluents, excipients, stabilizers, antiseptics, immuno-adjuvants, colors, flavors, and if necessary, physiologically active substances. The stabilizers include following examples: proteins such as serum albumen and gelatins; saccharides such as glucose, sucrose, lactose, maltose, trehalose, sorbitol, maltitol, mannitol, and lactitol; and buffers mainly composed of citrates, phosphates, or carbonates. The physiologically active substances usable in combination include following examples: anti-inflammatory agents such as aspirin, flufenamic acid, mefenamic acid, diclofenac, indomethacin, tolmetin, ibuprofen, ketoprofen, phenylbutazone, oxyphenbutazone, anti-inflammatory enzyme preparations, gold preparations, and chloroquine preparations; immunosuppressants such as FK506, cyclophosphamide, azathioprine, methotrexate, cyclosporin A, and adrenal cortical hormones; and further, antagonists against receptors for IL-18 and other cytokines, for example, antibodies including humanized antibodies respectively against interleukin-1-receptor protein, interleukin-2-receptor protein, interleukin-5-receptor protein, interleukin-6-receptor protein, interleukin-8-receptor protein, interleukin-12-receptor protein, and IL-18-receptor protein; antagonists respectively against TNF-α, TNF-β, interleukin-1-receptor, interleukin-5-receptor, interleukin-8-receptor, and IL-18-receptor; and antibodies including humanized antibodies respectively against interleukin-1, interleukin-2, interleukin-5, interleukin-8, interleukin-6, interleukin-8, interleukin-12, and interleukin-18.

The agent for susceptive diseases of this invention further includes pharmaceutics in the form for a single shot of medication. The pharmaceutics in such form contain the IL-18-binding protein, for example, in a content corresponding to multiples (up to fourfold) or divisor (not less than 1/40) of its single dosage, in a physically united formula suitable for medication. The formulae of such pharmaceutics include extracts, elixirs, capsules, granules, pills, ophthalmic ointments, suspensions, emulsions, plasters, suppositories, powders, spirits, tablets, syrups, infusions, decoctions, injections, replacement fluids, tinctures, ophthalmic solutions, troches, ointments, cataplasmas, aromatic waters, liniments, lemonades, fluid extracts, and lotions, and if necessary, nasal drops, nasal sprays, inhalations for lower airway, sustained release preparations for ophthalmic treatment, plastering tablets for tunica mucosa oris, and clysters. The agent for susceptive diseases of this invention can be administered orally and parenterally; both the administrations can effectively treat or prevent the susceptive diseases. The agent of this invention can be administered to patients usually in accordance with the symptom of each patient observed before and/or after treatment, for example, at a dosage for adult humans of about 1 μg/shot to 1 g/shot, usually, about 10 μg/shot to 100 mg/shot, with a frequency of 1 to 4 shot/day or 1 to 5 shot/week over 1 day to half a year through oral route or parenteral route such as intracutaneous, subcutaneous, intramuscular, and intravenous routes.

The DNAs encoding the IL-18-binding protein of this invention are useful also in so-called “gene therapies.” In conventional gene therapies, the DNA of this invention can be inserted into a viral vector such as retroviral vector, adenoviral vector, and adeno-associated-viral vector, or incorporated in a liposome such as cationic polymer and membrane-fused liposome, and in such form, the DNA can be directly injected into patients with diseases susceptive to the IL-18-binding protein. Alternatively, into lymphocytes collected from such patients, the DNA of this invention can be introduced in vitro, and the lymphocytes can be autografted to the patients. Thus the DNAs of this invention exhibit a distinguished efficacy in gene therapies for immunopathies such as autoimmune diseases, allergic diseases, and other diseases including liver disorders and nerve system disorders, as well as in suppression of rejection reactions and excessive immunoreactions associated with organ transplantation. General procedures for the gene therapies as above are detailed, for example, in “Jikken-Igaku-Bessatsu, Bio-manual Up Series, Idenshichiryo-no-Kisogijutsu (Basic Techniques for Gene Therapy),” edited by Takashi Shimada, Izumi Saito, and Toshiya Ozawa, published by Yodosha (1996).

The following are to explain the preferred embodiments of this invention in line with Examples, while these Examples can be variously modified by the level of techniques in this field. In view of this, this invention should not be restricted to these Examples only. In following Examples, IL-18-binding ability was judged by percent inhibition as a criteria determinable by the binding assay as follows.

As effector cells, cells expressing IL-18 receptor abundantly on the surface thereof are prepared by introduction of a DNA encoding IL-18 receptor into CHO-K1 cells (ATCC CRL-9618), derived from Chinese hamster ovary. As an assay medium, RPMI-1640 medium (pH 7.2) containing 0.1% (w/v) sodium azide, 0.1% (v/v) bovine serum albumin, and 100 mM N-2-hydroxyethylpiperazine-N′-2-ethane sulfonic acid is prepared. In a system for test, 50 μl of a test sample appropriately diluted with the assay medium is admixed with 50 μl of ¹²⁵I-labeled IL-18 appropriately diluted with the assay medium, and shaken at 4° C. for 1 hour. This mixture is then admixed with 50 μl of a suspension of the effector cells in the assay medium having a cell density of 1×10⁷ cells/ml, and shaken at 4° C. for another 1 hour. Thereafter, the resultant suspension of the effector cells is overlaid on 200 μl of a mixture of dibutyl phthalate and dioctyl phthalate (1:1 by volume) poured in 1.5-ml centrifugal tube, and then centrifuged at 4° C. for 5 minutes. The supernatant is removed by aspiration. The residual cells are cut out together with the tube, and measured for radio activity by gamma counter (“Type ARC-300,” produced by Aloka Co., Ltd.). Further, a system (for non-specific binding) in which 5 μg of non-labeled IL-18 is added together with ¹²⁵I-labeled IL-18 and another system (for total binding) with no test sample are treated similarly as in the test system. The measured radio activities, in the systems for test, total binding, and non-specific binding, are introduced into the following equation to calculate percent inhibition (%).

$$Percent Inhibition ( \% ) = \frac{( Total Binding ) - ( Test )}{( Total Binding ) - ( Non \text{-} Specific Binding )} \times 100$$

### EXAMPLE 1

### IL-18-Binding Protein of Human Origin

### Example 1-1

### Preparation of IL-18-Binding Protein

Three liters of human urine was concentrated with a membrane, and dialyzed against 20 mM phosphate buffer (pH 7.0) at 40 C for 20 hours. The dialyzed liquid was collected, and then applied to a column with 230 ml of affinity chromatography gel (“Wheat Germ Lectin Sepharose 6 MB,” commercialized by Amersham Pharmacia Biotech Co., Ltd.), which had been equilibrated with 20 mM phosphate buffer (pH 7.0), to adsorb the IL-18-binding protein. The column was washed with 20 mM phosphate buffer (pH 7.0), and 20 mM phosphate buffer (pH 7.0) containing 0.5 M N-acetyl-D-glucosamine was then fed to the column while the liquid eluted from the column was fractionated by a prescribed volume.

The eluted fractions were examined for IL-18-binding ability by the above-described binding assay. Fractions in which IL-18-binding property was observed were pooled and dialyzed against 20 mM phosphate buffer (pH 7.0) at 40 C for 16 hours. The dialyzed liquid was collected, concentrated to a prescribed volume, and then applied to a column with 54 ml of ion-exchange chromatography gel (“TSK-gel DEAE-5PW,” produced by TOSO Co., Ltd.), which had been equilibrated with 20 mM phosphate buffer (pH 7.0). To the column, 20 mM phosphate buffer (pH 7.0) containing sodium chloride was fed at a flow rate of 2 ml/min while the sodium chloride concentration was controlled to increase from 0 to 0.5 M over 100 minutes in a linear gradient manner. A fraction eluted at about 0.2 M sodium chloride was collected.

The above fraction was membrane-concentrated, and then applied to a column with 120 ml of gel-filtration chromatography gel (“HilLoad Superdex 200,” Amersham Pharmacia Biotech Co., Ltd.), which had been equilibrated with 20 mM phosphate-beffered saline (hereinafter abbreviated as “PBS”). To the column PBS was fed, and a fraction corresponding to a molecular weight of about 70,000 daltons on this gel filtration chromatography was collected. This newly obtained fraction was applied to a column with 4 ml of reversed phase chromatography gel (“Vydac 214TP54,” commercialized by Cypress International, Ltd.), which had been equilibrated with 0.1% (v/v) trifluoroacetic acid. To the column, 0.1% (v/v) trifluoroacetic acid containing acetonitrile was fed while the acetonitrile concentration was controlled to increase from 0 to 90% (v/v) in a linear gradient manner, and the liquid eluted from the column was fractionated by a prescribed volume. The eluted fractions were examined for IL-18-binding ability by the above-described binding assay. In fractions eluted at about 70% (v/v) acetonitrile, IL-18-binding ability was observed, and these fractions were pooled and concentrated. Thus a purified preparation of the IL-18-binding protein of human origin was obtained in a yield of about 3 μg.

This purified preparation of the IL-18-binding protein was examined for molecular weight by SDS-PAGE in the presence of dithiothreitol. A homogenous protein band bearing IL-18-binding ability was observed at the position of about 40,000 to 60,000 daltons. In addition, the IL-18-binding protein according to this Example was elucidated to be a glycoprotein by the fact that it adsorbed on “Wheat Germ Lectin Sepharose 6 MB” of which ligand is wheat germ lectin.

### Example 1-2

### N-Terminal Amino Acid Sequence

A purified preparation of the IL-18-binding protein, obtained by the method in Example 1-1, was dried up by a centrifugal concentrator, treated with 0.1 M Tris-HCl buffer (pH 8.1) containing 8 M urea and 10 mM EDTA under a current of nitrogen gas at 50° C. for 30 minutes, and reduced by an appropriate amount of dithiothreitol admixed therewith under a current of nitrogen gas at 50° C. for 2 hours. This reaction mixture was admixed with an appropriate amount of monoiodoacetic acid and reacted under dark conditions at ambient temperature for 30 minutes to alkylate the IL-18-binding protein.

The above-obtained, alkylated product was subjected to SDS-PAGE in the presence of dithiothreitol. A protein corresponding to a molecular weight of about 40,000 to about 60,000 daltons was separated, and transferred to a PDVF membrane. The membrane was subjected to amino acid analysis with protein sequencer (“Type 473A,” produced by Applied Biosystems) to determine the N-terminal amino acid sequence. The IL-18-binding protein of this invention according to Example 1-1 was proved to comprise the amino acid sequence shown in SEQ ID NO:3 (“Xaa” means an unidentified amino acid.) as the N-terminal amino acid sequence.

### Example 1-3

### Peptide Mapping

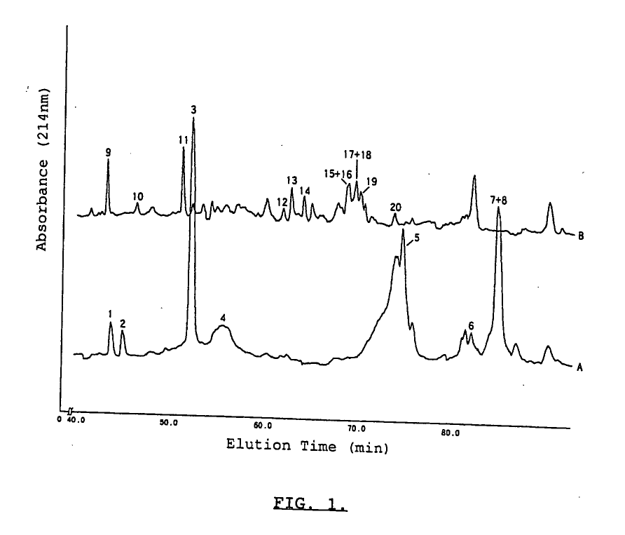

By the method “in-gel digestion” described in Ulf Hellman et al., “Analytical Biochemistry,” Vol. 224, pp. 451-455 (1995), peptide maps of the IL-18-binding protein were prepared from the IL-18-binding protein which was reduced and alkylated by the method in Example 1-2 and then digested with trypsin or trypsin-pepsin. Further, the trypsin-produced peptide fragments 1 to 8 and trypsin-pepsin-produced peptide fragments 9 to 20 were sequenced. The peptide fragments 1 to 20 were proved to have the amino acid sequences shown in SEQ ID NOs:4 to 23 (“Xaa” means an unidentified amino acid.), respectively. The above-prepared peptide maps are shown in FIG. 1.

### Example 1-4

### IL-18-Suppressive Activity

A test for IL-18-suppressive activity was conducted similarly as in Example 3-3, described below, except for using lymphocytes from a healthy human, recombinant human IL-18, and standard human IFN-γ (Gg02-901-530) obtained from National Institute of Health of U.S.A. as immunocompetent cells, IL-18, and IFN-γ standard, respectively.

The induction of IFN-γ production by the action of human IL-18 was significantly suppressed by the co-existence of the IL-18-binding protein according to Example 1. This indicates that this IL-18-binding protein suppresses the physiological activities of IL-18.

### EXAMPLE 2

### DNA Encoding IL-18-Binding Protein of Human Origin

### Example 2-1

### DNA Encoding IL-18-Binding Protein of Human Origin

### Example 2-1(a)

### Nucleotide Sequence of DNA Encoding IL-18-Binding Protein of Human Origin

Ten nanograms of human liver poly(A)⁺ RNA (product of Clontech) was mixed with 2 μl of 10×PCR buffer, 2 μl of 25 mM magnesium chloride, 2 μl of 0.1 M dithiothreitol, 1 μl of 25 mM DNTP mix, 1 μl of 200 units/1 μl reverse transcriptase (“Superscript II,” produced by Life-Tech Oriental Co., Ltd.), and 1 μl of 2.5 μM random hexamer, and the total volume was adjusted to 20 μl with sterilized-distilled water. This mixture was placed in a 0.5 ml reaction tube, and incubated sequentially at 42° C. for 50 minutes and 70° C. for 15 minutes to effect reverse transcriptase reaction. Thus a reaction product containing first strand cDNA was obtained.

This reaction product was admixed with 2.5-fold volumes of ethanol and 2 μl of 3 M sodium acetate, and allowed to stand at −20° C. for 2 hours to precipitate the cDNA. The precipitate was collected, washed with 75% (v/v) ethanol in water, dissolved in sterilized-distilled water, admixed with 0.5 μl of 2.5 units/μl DNA polymerase (“Cloned Pfu polymerase,” product of Stratagene), 10 μl of 25 mM dNTP mix, and further admixed with the oligonucleotide shown by 5′-ACNCCNGTNWSNCA-3′ (SEQ ID NO:52) as a sense primer, chemically synthesized on the basis of the amino acid sequence of SEQ ID NO:3, and the oligonucleotide shown by 5′-TGNGCNARNACNACRTG-3′ (SEQ ID NO:53) as an antisense primer, chemically synthesized on the basis of the amino acid sequence of SEQ ID NO: 8, both in a volume of 10 μM, and the total volume was adjusted to 100 μl with sterilized-distilled water. This mixture was incubated under 40 cycles of the sequential conditions at 94° C., 40° C., and 72° C. for 1 minute each to effect PCR.

A portion of the PCR product was collected and then electrophoresed on 1% (w/v) agarose gel to separate DNA fragments, and the DNA fragments were transferred to a nylon membrane and fixed thereon with 0.4 N sodium hydroxide. The membrane was washed with 2×SSC, dried in air, immersed in prehybridization solution containing 6×SSPE, 5× Denhardt's solution, 0.5% (w/v) SDS, and 100 μg/ml denatured salmon sperm DNA, and incubated at 65° C. for hours. A probe was prepared by chemical synthesis of the oligonucleotide shown by 5′-GGRCANGGRTCYTT-3′ (SEQ ID NO:54), based on the amino acid sequence shown in SEW ID NO:3, and isotope-labeling thereof with [γ-³²P]ATP by T4 polynucleotide kinase. To the pre-hybridization solution in which the above nylon membrane had been immersed, 1 μmol of the probe was added, and the nylon membrane was incubated at 40° C. for another 20 hours to effect hybridization. The nylon membrane was washed with 6×SSC and subjected to autoradiography in a usual manner. A specific hybridization signal by the nylon membrane was incubated at 40° C. for another 20 hours to effect hybridization. The nylon membrane was washed with 6×SSC and subjected to autoradiography in a usual manner. A specific hybridization signal by the probe was observed. This showed that the above PCR product contained the objective DNA fragment. To the remaining part of the above PCR product, 1 ng of a plasmid vector (“pCR-Script Cam SK(+),” produced by Stratagene) was added, and the DNA fragment of the PCR product was inserted into the vector with a DNA ligation kit (“DNA Ligation Kit, Version 2,” produced by Takara Shuzo Co., Ltd.). With a portion of the reaction mixture collected, an Escherichia coli strain (“XL1-Blue MRF' Kan,” produced by Stratagene) was transformed. The transformant was inoculated in LB medium (pH 7.5) containing 30 μg/ml chloramphenicol and cultured at 37° C. for 18 hours. The cells were collected from the culture. The plasmid DNA was collected from the cells in a usual manner, and analyzed by dideoxy method. This plasmid DNA comprised the nucleotide sequence shown in SEQ ID NO:34 as the sequence of the DNA fragment produced by PCR. The amino acid sequence encoded by this nucleotide sequence, aligned therewith, were compared with the partial amino acid sequences determined in Examples 1-2 to 1-3, shown in SEQ ID NOs:3 to 23. These partial amino acid sequences were completely or partly included by the amino acid sequence (SEQ ID NO:42) aligned in SEQ ID NO:34. This suggested that the nucleotide sequence shown in SEQ ID NO:34 encodes at least a part of the IL-18-binding protein of human origin.

### Example 2-1(b)

### Nucleotide Sequence Encoding IL-18-Binding Protein of Human Origin

Ten nanograms of human liver poly(A)⁺ RNA (product of Clontech) was subjected to 5′RACE, a modified method of PCR, with a commercially available 5′RACE kit (“5 RACE System, Version 2.0,” product of GIBCO BRL). First, reverse transcriptase reaction was effected on the above RNA with the oligonucleotide shown by 5′GGTCACTTCCAATGCTGGACA-3′ (SEQ ID NO:55) as a primer, chemically synthesized on the basis of the nucleotide sequence shown in SEQ ID NO:34, and to the 5′-terminal of the first strand cDNA synthesized thereby, C-tail was added by the action of terminal deoxynucleotidyl transferase. Then, PCR was effected on this first strand cDNA with the oligonucleotide shown by 5′-GGCCACGCGTCGACTAGTACGGGIIGGGIIGGGIIG-3′ (SEQ ID NO:56) as a sense primer, included by the above kit, and the oligonucleotide shown by 5′-GTCCTTTGTGCTTCTAACTGA-3′ (SEQ ID NO:57) as an antisense primer, chemically synthesized on the basis of the nucleotide sequence shown in SEQ ID NO:34. A portion of the produce of this 5′ RACE was collected, and electrophoresed in a usual manner on 1% (w/v) agarose. Specific amplification of a DNA fragment was observed. This DNA fragment was sequenced similarly as in Example 2-1(a). This fragment comprised the nucleotide sequence shown in SEQ ID NO:35. The sequence from the 160^{th} to 216^{th} nucleotides of this sequence completely matched with the sequence from the 1^{st} to 57^{th} nucleotides of the nucleotide sequence shown in SEQ ID NO:34, determined in Example 2-1(a). This suggested that the nucleotide sequence shown in SEQ ID NO: 35 overlaps with the nucleotide shown in SEQ ID NO:34, encoding at least a part of the IL-18-binding protein (SEQ ID NO:43) of human origin, and comprises the 5′-upsteam region of SEQ ID NO:34.

### Example 2-1(c)

### Nucleotide Sequence Encoding IL-18-binding protein of Human Origin

Ten nanograms of human liver poly(A)⁺ RNA was subjected to 3′RACE, a modified method of PCR, in accordance with “PCR Jikken Manual (Manual for PCR Experiments),” transplanted by Takashi Saito, published by HBJ Press (1991), 25-33. First, reverse transcriptase reaction was effected on the above RNA with the oligonucleotide shown by 5′-GACTCGAGTCGACATCGA(T)₁₇-3′ (SEQ ID NO:58) as a primer. Then, PCR was effected on the first strand cDNA synthesized thereby with the oligonucleotide shown by 5′-TTCTCCTGTGTGCTCGTGGA-3′ (SEQ ID NO:59) as a sense primer, chemically synthesized on the basis of the nucleotide sequence shown in SEQ ID NO:34, determined in Example 2-1(a), and the oligonucleotide shown by 5′-GACTCGAGTCGACATCG-3′ (SEQ ID NO:60) as an antisense primer. A portion of the product of this 3′RACE was collected and electrophoresed in a usual manner on 1% (w/v) agarose. Specific amplification of a DNA fragment was observed. This DNA fragment was sequenced similarly as in Example 2-1(a). This fragment comprised the nucleotide sequence shown in SEQ ID NO:36. The sequence from the 1^{st} to 60^{th} nucleotides of this sequence completely matched with the sequence from the 352^{nd} to 411st nucleotides of the nucleotide sequence shown in SEQ ID NO:34, determined in Example 2-1(a). This suggested that the nucleotide sequence shown in SEQ ID NO:36 (SEQ ID NO:44) overlaps with the nucleotide sequence shown in SEQ ID NO:34, encoding at least a part of the IL-18-binding protein of human origin, and comprises the 3′-downstream region of SEQ ID NO:34.

As described above, in Examples 2-1(a) to 2-1(c), the nucleotide sequences shown in SEQ ID NOs:34 to 36 were determined as ones partially encoding the IL-18-binding protein of human origin and overlapping one another. In view of the overlapping sequences, these three nucleotide sequences would be derived from one contiguous nucleotide sequence, which is shown in SEQ ID NO:37.

### Example 2-1(d)

### Nucleotide Sequence of DNA Encoding Human-Derived IL-18-Binding Protein

In accordance with the method in Example 2-1(a), reverse transcriptase reaction was effected on human liver poly(A)⁺ RNA, and then PCR was effected similarly as in Example 2-1(a) except for using as a sense primer the oligonucleotide shown by 5′-TGTGTGACTGGAGAAGAGGAC-3′ (SEQ ID NO:50), chemically synthesized on the basis of the nucleotide sequence shown in SEQ ID NO:37, and as an antisense primer the oligonucleotide shown by 5′-TACAGGCAGTCAGGGACTGTTCACTCCAG-3′ (SEQ ID NO:51), chemically synthesized on the basis of the nucleotide sequence shown in SEQ ID NO:37. A portion of the PCR product was collected, and electrophoresed in a usual manner on 1% (w/v) agarose gel. Specific amplification of a DNA fragment was observed. This DNA fragment was sequenced similarly as in Example 2-1(a). This fragment comprised the nucleotide sequence shown in SEQ ID NO:37. This supported that the nucleotide sequences shown in SEQ ID NOs:34 to 36, determined in Examples 2-1(a) to 2-1(c), are partial sequences of the contiguous nucleotide sequence shown in SEQ ID NO:37.

The amino acid sequence (SEQ ID NO:45) encoded by the nucleotide sequence shown in SEQ ID NO:37, aligned therewith, are compared with the partial amino acid sequences shown in SEQ ID NOs:4 to 23, determined in Example 1-3. These partial sequences were all included by the amino acid sequence aligned in SEQ ID NO:37 in the region from the 1st to 164th amino acids. In addition, the N-terminal amino acid sequence determined in Example 1-2, shown in SEQ ID NO:3, well matched with the amino acid sequence aligned in SEQ ID NO:37 in the region from the 1st to 22nd amino acids. These facts suggested that the nucleotide sequence shown in SEQ ID NO:37 can encode the IL-18-binding protein of human origin by the region from the 160th to 651st nucleotides and that this IL-18-binding protein may has, as its whole sequence, the sequence from the 1st to 164th amino acids of the amino acid sequence aligned with this nucleotide sequence. Thus suggested amino acid sequence of the IL-18-binding protein of human origin and the nucleotide sequence encoding this are shown in SEQ ID NOs:1 and 32 separately.

### Example 2-2

### Production of IL-18-Binding Protein of Human Origin by Transformant

### Example 2-2(a)

### Preparation of Recombinant DNA

A DNA capable of encoding the IL-18-binding protein of human origin, obtained by the method in Example 2-1(d), was placed in a 0.5-ml reaction tube in an amount of 1 ng, and to this tube, 10 μl of 10×PCR buffer, 1 μl of 25 mM dNTP mix, and 2.5 units/μl DNA polymerase (“Cloned Pfu polymerase,” produced by Stratagene) were added. Appropriate amounts of the oligonucleotide shown by 5′-CTCGAGGCCACCATGACCATGAGACACAAC-3′ (SEQ ID NO:61) as a sense primer, chemically synthesized on the basis of the nucleotide sequence shown in SEQ ID NO:32, and the oligonucleotide shown by 5′-GCGGCCGCTCATTAGTGATGGTGATGGTGATGACCCTGCTGCTGTGGACT-3′ (SEQ ID NO:62) as an antisense primer, chemically synthesized on the basis of the nucleotide sequence shown in SEQ ID NO:32, were further added to the above tube, and the total volume was adjusted to 100 μl with sterilized-distilled water. PCR was effected by incubating this mixture under 3 cycles of the sequential conditions at 94° C. for 1 minute, at 42° C. for 2 minutes, and at 72° C. for 3 minutes and then 35 cycles of the sequential conditions at 94° C. for 1 minute, at 60° C. for 2 minutes, and 72° C. for 3 minutes. The PCR product was analyzed and manipulated similarly as in Example 2-1(a); the PCR product was confirmed to contain the objective DNA fragment, and a plasmid vector inserted with this DNA fragment was obtained. This plasmid DNA comprised the nucleotide sequence shown in SEQ ID NO:32, confirmed by sequencing similarly as in Example 2-1(a).

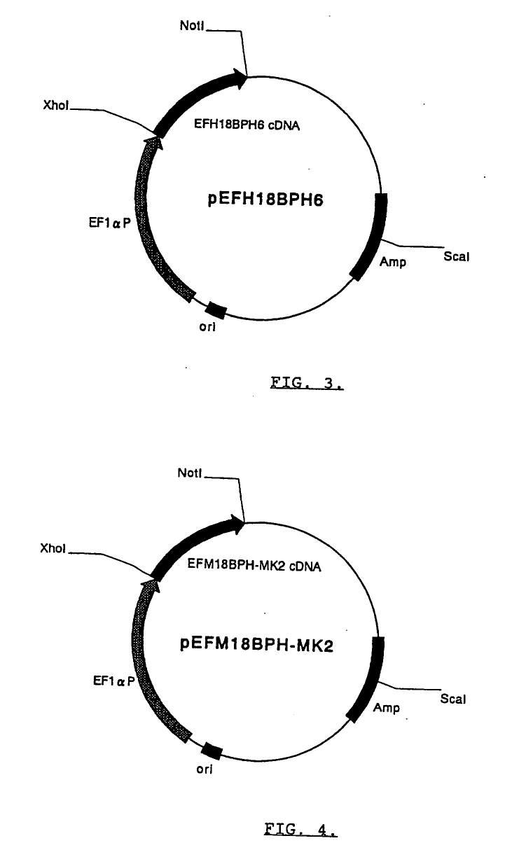

The restriction enzymes XhoI and NotI were allowed to react in a usual manner on the above plasmid DNA to produce a DNA fragment. This DNA fragment was mixed with the plasmid vector “pEF-BOS”, prepared similarly as in S. Mizushima et al., “Nucleic Acid Research,” Vol. 17, No. 18, p. 5332 (1990) and digested with XhoI and NotI, at their proportion of 100 ng to 10 ng, and the DNA fragment was inserted into the plasmid vector with a DNA ligation kit (“DNA Ligation Kit, Version 2,” produced by Takara Shuzo Co., Ltd.). Similarly as in Example 2-1(a), the Escherichia coli strain was transformed with this ligation product. From the resultant transformant, the recombinant DNA was collected, and named “pEFH18BPH6.” This recombinant DNA was analyzed in a usual manner. As shown in FIG. 3, in the recombinant DNA “pEFH18BPH6,” the cDNA “EFH18BPH6 cDNA” comprising the nucleotide sequence shown in SEQ ID NO:32, capable of encoding the IL-18-binding protein of human origin, was located on the downstream of the elongation factor 1 promotor “EF1αP.”

### Example 2-2(b)

### Production of IL-18-Binding Protein of Human Origin by Transformant

The Escherichia coli strain transformed with the recombinant DNA “pEFH18BPH6” in Example 2-2(a) was inoculated in LB broth (pH 7.2) containing 100 μg/ml ampicillin, and cultured at 37° C. under aerobic conditions by agitation. From the resultant culture, the plasmid DNA was collected in a usual manner to obtain the recombinant DNA “pEFH18BPH6”. Twenty micrograms of this recombinant DNA was introduced by electroporation into 1×10⁷ cells of COS-1 (ATCC CRL-1650), a fibroblastic cell line derived from African green monkey kidney, which had been proliferated in a usual manner. Thus a transformant introduced with the DNA of this invention was obtained.

A medium (“ASF104,” product of Ajinomoto) was placed in flat-bottomed culture flasks. The above-obtained transformant was inoculated into the medium at a ratio of 1×10⁵ cells/ml, and cultured in a 5% CO₂ incubator at 37° C. for 3 days. The culture supernatant was collected from the resultant culture, and applied to a column with affinity chromatography gel (“Ni-NTA,” product of QIAGEN). PBS containing 20 mM imidazole was fed to the column to remove non-adsorbed fraction, and then PBS containing 250 mM imidazole was fed while the liquid eluted from the column was fractionated by a prescribed volume. These fractions were examined for IL-18-binding ability by the above-described binding assay. Fractions with IL-18-binding ability were pooled. Thus an aqueous solution of purified IL-18-binding protein was obtained in a volume of about 2 ml. This solution contained about 10 μg/ml protein. After this solution was treated similarly as in Example 1-2, the N-terminal amino acid sequence was analyzed. The elucidated sequence was identical with the amino acid sequence shown in SEQ ID NO:3. As a control, procedures similar to this Example were conducted by using the plasmid vector “pEF-BOS” in place of the recombinant DNA “pEFH18BPH6.” No IL-18-binding protein was observed. These results supported that the IL-18-binding protein of human origin usually has the amino acid sequence shown in SEQ ID NO:1 and can be encoded by the nucleotide sequence shown in SEQ ID NO:32.

### EXAMPLE 3

### IL-18-Binding Protein of Mouse Origin

### Example 3-1

### Preparation of IL-18-Binding Protein

Corynebacterium parvum (ATCC 11827) was heated at 60° C. for 1 hour. The dead cells thus obtained were injected with needles into 600 heads of 8-week-old, female CD-1 mice at a dose of 1 mg/head through intraperitoneal routes. The mice were housed in a usual manner for 7 days, and then injected with purified Escherichia coli lipopolysaccharide through intravenous routes at a dose of 1 μg/head. Two hours later, the blood was collected from the mice's hearts, and by usual manipulation, 200 ml of serum was obtained from the blood. The serum was subjected to purification by the method in Example 1-1. Thus a purified preparation of the IL-18-binding protein of mouse origin was obtained in a yield of about 3 μg.

This purified preparation was examined for molecular weight by SDS-PAGE in the presence of dithiothreitol. A homogenous protein band bearing IL-18-binding ability was observed at the position of about 40,000 to 60,000 daltons. In addition, the IL-18-binding protein according to this Example was elucidated to be a glycoprotein by the fact that it adsorbed on “Wheat Germ Lectin Sepharose 6 MB” of which ligand is wheat germ lectin.

### Example 3-2

### Peptide Mapping

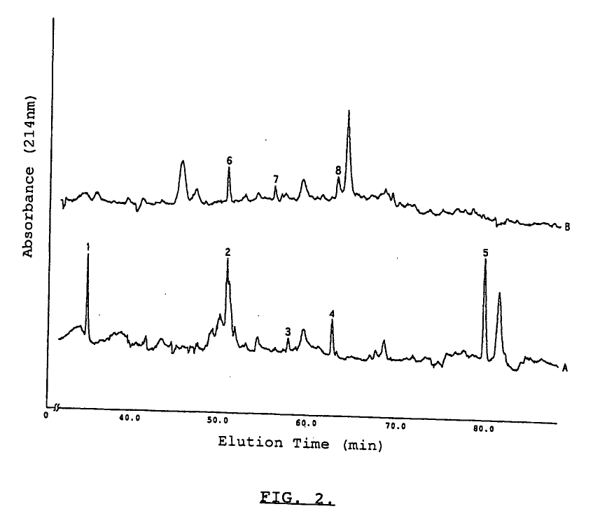

Similarly as in Example 1-3, peptide maps were prepared from a purified preparation of the IL-18-binding protein, obtained by the method in Example 3-1, and amino acid sequences were analyzed on the trypsin-produced peptide fragments 1 to 5 and trypsin-pepsin-produced peptide fragments 6 to 8. The peptide fragments 1 to 8 were proved to have the amino acid sequences shown in SEQ ID NOs:24 to 31 (“Xaa” means an unidentified amino acid.), respectively. The above-prepared peptide maps are shown in FIG. 2.

### Example 3-3

### IL-18-Suppressive Activity

Spleens were extracted from 14-week-old, female C3H/HeJ mice, and dispersed. After the adherent cells were removed, the spleen cells were suspended to use as immunocompetent cells in RPMI-1640 medium (pH 7.4) supplemented with 10% (v/v) fetal calf serum. The spleen cell suspension and 2.5 μg/ml concanavalin A were distributed to microplates at 0.15 ml and 0.05 ml per well. To each well, the above medium containing 25 ng/ml recombinant mouse IL-18 and a purified preparation of the IL-18-binding protein, prepared by the method in Example 3-1, at a content excessive to the IL-18, was added in a volume of 0.05 ml/well. The microplates were incubated in a 5% CO₂ incubator at 37° C. for 24 hours. After the culture, 0.1 ml portion of each culture supernatant was collected, and measured for IFN-γ production by conventional enzyme-immunoassay. As controls, systems with no IL-18-binding protein or no mouse IL-18 were treated similarly as above. The measured values of IFN-γ were converted into international units (IU) with reference to the standard mouse IFN-γ (Gg02-901-533) obtained from National Institute of Health, U.S.A., as an IFN-γ standard.

IFN-γ produced in the control with no IL-18-binding protein was about 600 IU/ml, and that in the other control, with no mouse IL-18, was 0 IU/ml. In the test system with IL-18-binding protein, IFN-γ was produced only about 60 IU/ml. These results indicated that the IL-18-binding protein according to Example 3 suppresses the physiological activities of IL-18.

### EXAMPLE 4

### DNA encoding IL-18-Binding Protein of Mouse Origin

### Example 4-1

### DNA Encoding IL-18-Binding Protein of Mouse Origin

### Example 4-1(a)

### Nucleotide Sequence of DNA Encoding IL-18-Binding Protein of Mouse Origin

Corynebacterium parvum (ATCC 11827) was heated at 60° C. for 1 hour. The dead cells thus obtained were injected with needles into 8-week-old, female CD-1 mice at a dose of 1 mg/head through intraperitoneal routes. The mice were housed in a usual manner for 7 days, and then injected with purified Escherichia coli lipopolysaccharide through intravenous routes at a dose of 1 μg/head. Two hours later, the mice were slaughtered by dislocating each tibia, and the livers were extracted. Three grams by wet weight of the livers were immersed in 20 ml of a liquid (pH 7.0) consisting of 6 M guanidine isothiocyanato, 10 mM sodium citrate, and 0.5% (w/v) SDS, and disrupted with a homogenizer. In 35-ml centrifugal tubes, 0.1 M EDTA (pH 7.5) containing 5.7 M cesium chloride was poured in a volume of 25 ml/tube, and the cell disruptant was overlaid thereon at 10 ml/tube and then ultracentrifuged at 25,000 rpm for 20 hours at 20_C. The RNA fraction was collected, placed in a 15-ml centrifugal tube, and admixed with an equal volume of chloroform-isobutanol (4:1 by volume). The mixture was shaken for 5 minutes and centrifuged at 10,000 rpm for 10 minutes at 40 C, and the resultant liquid layer was collected. The liquid layer was admixed with 2.5-fold volumes of ethanol and allowed to stand at −20° C. for 2 hours to precipitate total RNA. The precipitate was collected, washed with 75% (v/v) ethanol in water, and dissolved in 0.5 ml of sterilized-distilled water.

Reverse transcriptase reaction was effected similarly as in Example 2-1(a) on this total RNA, and PCR was effected on this reaction product containing first strand cDNA similarly as in Example 2-1(a) except for using as a sense primer the oligonucleotide shown by 5′-GCNGTNCCNACNAA-3′ (SEQ ID NO:63), chemically synthesized on the basis of the amino acid sequence shown in SEQ ID NO: 27, and as an antisense primer the oligonucleotide shown by 5′-GTYTTNARNCCRTC-3′ (SEQ ID NO:64), chemically synthesized on the basis of the amino acid sequence shown in SEQ ID NO:30. A probe was prepared from the oligonucleotide shown by 5′-SWNCTRTGNCCYTCYTT-3′ (SEQ ID NO:65), chemically synthesized on the basis of the amino acid sequence shown in SEQ ID NO:24. By using this probe and by the procedure according to Example 2, the above PCR product was confirmed to contain the objective DNA fragment. This DNA fragment was sequenced similarly as in Example 2-1(a). This fragment comprised the nucleotide sequence shown in SEQ ID NO:38. The amino acid sequence (SEQ ID NO:46) aligned in SEQ ID NO:38 was compared with the partial amino acid sequences shown in SEQ ID NOs:24 to 31, determined in Example 3-2. These partial amino acid sequences were completely or partly included by the amino acid sequence aligned in SEQ ID NO: 38. This suggested that the nucleotide sequence shown in SEQ ID NO:38 encodes at least a part of the IL-18-binding protein of mouse origin.

### Example 4-1(b)

### Nucleotide Sequence of DNA Encoding IL-18-Binding Protein of Mouse Origin

Total RNA was collected similarly as in Example 4-1(a) from female CD-1 mice treated with the dead cells of Corynebacterium parvum and lipopolysaccharide, and 1 μg of the total RNA was subjected to 5′RACE, a modified method of PCR, with a commercially available 5′ RACE kit (“5′RACE System, Version 2.0,” product of GIBCO BRL). First, reverse transcriptase reaction was effected on the above total RNA with the oligonucleotide shown by 5′-TGCAGGCAGTACAGGACAAGG-3′ (SEQ ID NO: 66) as a primer, chemically synthesized on the basis of the nucleotide sequence shown in SEQ ID NO:38, and to the 5′-terminal of the first strand cDNA synthesized thereby, C-tail was added by the action of terminal deoxynucleotidyl transferase. Then, PCR was effected on this first strand cDNA with the oligonucleotide shown by 5′-GGCCACGCGTCGACTAGTACGGGIIGGGIIGGGIIG-3′ (SEQ ID NO:56) as a sense primer, included by the kit, and the oligonucleotide shown by 5′-GTGCTGGGTACTGCTTAGTTG-3′ (SEQ ID NO:67) as an antisense primer. A portion of this 5′RACE product was collected, and electrophoresed in a usual manner on 1% (w/v) agarose gel. Specific amplification of a DNA fragment was observed. This DNA fragment was sequenced similarly as in Example 2-1(a). This fragment comprised the nucleotide sequence shown in SEQ ID NO:39. The sequence from the 307th to 336th nucleotides of this sequence completely matched with the sequence of the 1st to 30th nucleotides of the sequence shown in SEQ ID NO:38, determined in Example 4-1(a). This suggested that the nucleotide sequence shown in SEQ ID NO:39 overlaps with the nucleotide sequence shown in SEQ ID NO:38, encoding at least a part of the IL-18-binding protein (SEQ ID NO:47) of mouse origin, and comprises the 5′-upstream region of SEQ ID NO:38.

### Example 4-1(c)

### Nucleotide Sequence of DNA Encoding IL-18-Binding Protein of Mouse Origin

Total RNA was collected similarly as in Example 4-1(a) from female CD-1 mice treated with the dead cells of Corynebacterlum parvum and lipopolysaccharide, and 1 μg of the total RNA was subjected to 3′RACE, a modified method of PCR, in accordance with the methods described in “PCR Jjkkeri Manual (Manual for PCR Experiments),” translated by Takashi Saito, published by HBJ Press (1991), pp. 25-33. First, reverse transcriptase reaction was effected on the above total RNA with the oligonucleotide shown by 5′-GACTCGAGTCGACATCGA(T)₁₇-3′ (SEQ ID NO:58) as a primer. Then, PCR was effected on the first strand cDNA synthesized thereby with the oligonucleotide shown by 5′-GATCCTGGACAAGTGGCC-3′ (SEQ ID NO:68) as a sense primer, chemically synthesized on the basis of the nucleotide sequence shown in SEQ ID NO:38, determined in Example 4-1(a), and the oligonucleotide shown by 5′-GACTCGAGTCGACATCG-31 (SEQ ID NO:60) as an antisense primer. A portion of this 3′RACE product was collected, and electrophoresed in a usual manner on 1% (w/v) agarose gel. Specific amplification of a DNA fragment was observed. This DNA fragment was sequenced similarly as in Example 2-1(a). This fragment comprised the nucleotide sequence shown in SEQ ID NO:40. The sequence from the 1st to 63rd nucleotides of this sequence completely matched with the sequence of the 289th to 351st nucleotides of the sequence shown in SEQ ID NO:38, determined in Example 4-1(a). This suggested that the nucleotide sequence shown in SEQ ID NO:40 overlaps with the nucleotide sequence shown in SEQ ID NO:38, encoding at least a part of the IL-18-binding protein (SEQ ID NO:48) of mouse origin, and comprises the 3′-downstream region of SEQ ID NO:38.

As described above, in Examples 4-1(a) to 4-1(c), the nucleotide sequences shown in SEQ ID NOs:38 to 40 were determined as ones partially encoding the IL-18-binding protein of mouse origin and overlapping one another. In view of the overlapping sequences, these three nucleotide sequences would be derived from one contiguous nucleotide sequence, which is shown in SEQ ID NO:41.

### Example 4-1(d)

### Nucleotide Sequence of DNA Encoding IL-18-Binding Protein of Mouse Origin

Total RNA was collected similarly as in Example 4-1(a) from female CD-1 mice treated with the dead cells of Corynebacterium parvum and lipopolysaccharide. After reverse transcriptase reaction was effected on this total RNA, PCR was effected similarly as in Example 4-1(c) except for using the oligonucleotide shown by 5′-CTGAGCCTTAGAGCTCCAAG-3′ (SEQ ID NO:69) as a sense primer and the oligonucleotide shown by 5′-GTGAAGCTTGAGTTTGAGGTTC-3′ (SEQ ID NO:70) as an antisense primer, both chemically synthesized on the basis of the nucleotide sequence shown in SEQ ID NO:41. A portion of this PCR product was collected, and electrophoresed in a usual manner on 1% (w/v) agarose gel. Specific amplification of a DNA fragment was observed. This DNA fragment was sequenced similarly as in Example 2-1(a). This fragment comprised the nucleotide sequence shown in SEQ ID NO:41. The amino acid sequence (SEQ ID NO:49) encoded by the nucleotide sequence shown in SEQ ID NO:41, aligned therewith, are compared with the partial amino acid sequences shown in SEQ ID NOs:24 to 31, determined in Example 3-2. These partial sequences were all included by the amino acid sequence aligned in SEQ ID NO:41 in the region from the 1st to 165th amino acids. In addition, the amino acid sequence of the IL-18-binding protein of human origin shown in SEQ ID NO:1 exhibited about 61% homology with the amino acid sequence aligned in SEQ ID NO:41 in the region from the 1st to 165th amino acids. These facts suggested that the nucleotide sequence shown in SEQ ID NO:41 can encode the IL-18-binding protein of mouse origin by the region from the 235th to 729th nucleotides and that this IL-18-binding protein may have, as its whole sequence, the sequence from the first to 165th amino acids of the amino acid sequence aligned with this nucleotide sequence. The amino acid sequence thus suggested as that of the IL-18-binding protein of mouse origin and the nucleotide sequence encoding this are shown in SEQ ID NOs:2 and 33 separately.

### Example 4-2

### Production of IL-18-Binding Protein of Mouse Origin by Transformant

### Example 4-2(a)

### Preparation of Recombinant DNA

A DNA capable of encoding the IL-18-binding protein of mouse origin, obtained by the method in Example 4-1(d), was placed in a 0.5-ml reaction tube in an amount of 1 ng, and this DNA was treated similarly as in Example 2-2(a) except for using the oligonucleotide shown by 5′-CTCGACGCCACCATGACCATGAGACACTGC-3′ (SEQ ID NO:71) as a sense primer and the oligonucleotide shown by 5′-GCGGCCGCTCATTAGTGATGGTGA TGGTGATGTGCAACCCCTGGGCCTGC-3′ (SEQ ID NO:72), as an antisense primer, both on the basis of the nucleotide sequence shown in SEQ ID NO:33. Similarly as in Example 4-1(a), the PCR product was confirmed to contain the objective DNA fragment, and a plasmid vector inserted with this DNA fragment was obtained. This plasmid DNA was sequenced similarly as in Example 2-1(a). The plasmid DNA comprised the nucleotide sequence shown in SEQ ID NO:33.

DNA insertion was effected from the above-obtained plasmid DNA into the plasmid vector “pEF-BOS” similarly as in Example 2-2(a). Thus obtained recombinant DNA was named “pEFM18BPH-MK2.” This recombinant DNA was analyzed in a usual manner. As shown FIG. 4., in the recombinant DNA “pEFM18BPH-MK2,” the cDNA “EFM18BPH-MK2 cDNA” comprising the nucleotide sequence shown in SEQ ID NO:33, capable of encoding the IL-18-binding protein of mouse origin, was located on the downstream of the elongation factor 1 promotor “EF1αP.”

### Example 4-2(b)

### Production of IL-18-Binding Protein of Mouse Origin by Transformant

From the culture of the Escherichia coli strain transformed with the recombinant DNA “pEFM18BPH-MK2” in Example 4-2, the plasmid DNA was collected in a usual manner to obtain the recombinant DNA “pEFM18BPH-MK2.” Twenty micrograms of this recombinant DNA was introduced into COS-1 cells (ATCC CRL-1650) similarly as in Example 2-2(b). Thus a transformant introduced with the DNA of this invention was obtained.

Similarly as in Example 2-2(b), the above transformant was cultured, and the culture supernatant was collected and fractionated through a column with affinity chromatography gel (“Ni-NTA,” product of QIAGEN). Fractions in which IL-18-binding protein was observed were collected and pooled. Thus an aqueous solution of purified IL-18-binding protein was obtained in a volume of about 2 ml from 1×10⁷ cells of the transformant. This solution contained about 1 μg/ml protein. After this solution was treated according to Example 1-2, the N-terminal amino acid sequence was analyzed. The elucidated sequence was identical with the amino acid sequence shown in SEQ ID NO:2. As a control, procedures similar to this Example were conducted by using the plasmid vector “pEF-BOS” in place of the recombinant DNA “pEFH18BPH6.” No IL-18-binding protein was observed. These results supported that the IL-18-binding protein of mouse origin usually has the amino acid sequence shown in SEQ ID NO:2 and can be encoded by the nucleotide sequence shown in SEQ ID NO:33.

The following are to explain the agent for susceptive disease containing the IL-18-binding protein of this invention as an effective ingredient.

### EXAMPLE 5

### Solution

A purified preparation of the IL-18-binding protein, obtained by the method in Example 1-1 or 2-2, was dissolved to give a concentration of 1 mg/ml in physiological saline containing as a stabilizer 1% (w/v) pulverized crystalline trehalose (“Trehaose,” commercialized by Hayashibara Shoji, Inc.) free from pyrogen. These solutions were made germ free in a usual manner. Thus two types of solutions were obtained.

These products, having excellent stability, are useful as an injection, ophthalmic solution, collunarium, etc. to treat or prevent the susceptive diseases including autoimmune diseases, inflammatory diseases, and allergic diseases.

### EXAMPLE 6

### Dried Injection

A purified preparation of the IL-18-binding protein, obtained by the method in Example 1-1 or 2-2, was dissolved at a ratio of 100 mg to 100 ml in physiological saline containing as a stabilizer 1% (w/v) sucrose free from pyrogen. These solutions were made germ free in a usual manner, distributed by 1 ml into vials, and lyophilized, and the vials were sealed.

These products, having excellent stability, are useful as a dried injection to treat or prevent the susceptive diseases including autoimmune diseases, inflammatory diseases, and allergic diseases.

### EXAMPLE 7

### Ointment

Carboxyvinyl polymer (“Hi-Bis Wako,” produced by Wako Pure Chemical Co., Ltd.) and pulverized crystalline trehalose (“Trehaose,” commercialized by Hayashibara Shoji, Inc.) free from pyrogen were dissolved in sterilized-distilled water to give the respective concentrations of 1.4% (w/w) and 2.0% (w/w). This solution was mixed to a homogeneity with a purified preparation of the IL-18-binding protein, obtained by the method in Example 1-1 or 2-2, and then adjusted to pH 7.2. Thus 2 types of paste containing about 1 mg/g IL-18-binding protein were obtained.

These products, having excellent spreadability and stability, are useful as an ointment to treat or prevent the susceptive diseases including autoimmune diseases, inflammatory diseases, and allergic diseases.

### EXAMPLE 8

### Tablets

Pulverized anhydrous maltose (“Finetose,” commercialized by Hayashibara Shoji, Inc.) free from pyrogen was mixed to homogeneity with a purified preparation of IL-18-binding protein, obtained by the method in Example 1-1 or 1-2, and Lumin as a cell activator. These mixtures were tableted in a usual manner so that two types of tablets, each piece (about 200 mg) containing about 1 mg of the IL-18-binding protein and about 1 mg of Lumin (produced by Nihon Kanko Shikiso Co., Ltd.), were obtained.

These products, having excellent ingestibility and stability as well as cell-activating activity, are useful as tablets to treat or prevent the susceptive diseases including autoimmune diseases, inflammatory diseases, and allergic diseases.

Experiment: Acute Toxicity Test

A purified preparations of the IL-18-binding protein, obtained by the method in Example 1-1,2-2, 3-1, or 4-2 was administered orally, intraperitoneally, or intravenously to five-week-old ddy mice (body weight of 20 to 25 g) in a usual manner. These purified preparations of the IL-18-binding protein had LD50 of about 1 mg/mouse-body-weight or higher, through any administration route. This indicates that it is safe to incorporate the IL-18-binding protein of this invention into pharmaceuticals to be administered to humans and other mammals.

### INDUSTRIAL APPLICABILITY

As described above, this invention is established on the basis of the finding of a novel protein which binds to IL-18. The protein of this invention suppresses the physiological activities of IL-18, which is responsible for activation of immune system, in humans and other mammals, and this protein exhibits a distinguished efficacy in alleviating rejection reactions associated with organ transplantation and in treating and preventing various diseases resulting from augmented immunoreactions.

1. A method for treating or preventing interleukin 18-susceptive diseases, which comprises administering an effective amount of either a protein or its fragment capable of binding and neutralizing interleukin 18 to a patient with a disease where interleukin 18 is responsible for its incidence, said protein having the amino acid sequence of SEQ ID NO:1.

2. The method of claim 1 wherein said interleukin 18-susceptive diseases are immunopathies and disorders in circulatory and nerve systems.

3. The method of claim 2, wherein said immunopathies are rejection reactions and allergic reactions.

4. The method of claim 1, wherein said protein is orally or parenterally administered to said subject at a dose of about 1 μg/shot to 1 g/shot for an adult human.

5. The method of claim 4, wherein said protein is administered to said subject at a frequency of 1 to 4 shots/day or 1 to 5 shots/week.


SEQUENCE LISTING (72 sequences)

SEQ ID NO: 1 - protein, 164 aa, Homo sapiens
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 2 - protein, 165 aa, Mus musculus
TSAPQTTATVLTGSSKDPCSSWSPAVPTKQYPALDVIWPEKEVPLNGTLTLSCTACSRFPYFSILYWLGNGSFIEHLPGRLKEGHTSREHRNTSTWLHRALVLEELSPTLRSTNFSCLFVDPGQVAQYHIILAQLWDGLKTAPSPSQETLSSHSPVSRSAGPGVA

SEQ ID NO: 3 - protein, 22 aa, Homo sapiens
  misc_feature (6)..(8): “Xaa” means an unidentified amino acid.
TPVSQXXXAAXAXVRXXKDPCP

SEQ ID NO: 4 - protein, 9 aa, Homo sapiens
GSTGTQLCK

SEQ ID NO: 5 - protein, 11 aa, Homo sapiens
ERGSTGTQLCK

SEQ ID NO: 6 - protein, 8 aa, Homo sapiens
LWEGSTSR

SEQ ID NO: 7 - protein, 15 aa, Homo sapiens
  misc_feature (6)..(8): “Xaa” means an unidentified amino acid.
TPVSQXXXAAXAXVR

SEQ ID NO: 8 - protein, 23 aa, Homo sapiens
  misc_feature (14): “Xaa” means an unidentified amino acid.
HVVLAQLWAGLRAXLPXXQEALP

SEQ ID NO: 9 - protein, 10 aa, Homo sapiens
  misc_feature (8)..(9): “Xaa” means an unidentified amino acid.
ALVLEQLXXA

SEQ ID NO: 10 - protein, 29 aa, Homo sapiens
  misc_feature (13)..(15): “Xaa” means an unidentified amino acid.
ALVLEQLTPALHXXXFXXVLVDPEQVVQR

SEQ ID NO: 11 - protein, 12 aa, Homo sapiens
  misc_feature (5): “Xaa” means an unidentified amino acid.
QCPAXEVTWXEV

SEQ ID NO: 12 - protein, 7 aa, Homo sapiens
WEGSTSR

SEQ ID NO: 13 - protein, 6 aa, Homo sapiens
LVDPEQ

SEQ ID NO: 14 - protein, 7 aa, Homo sapiens
IEHLPGR

SEQ ID NO: 15 - protein, 4 aa, Homo sapiens
HVVL

SEQ ID NO: 16 - protein, 7 aa, Homo sapiens
EQLTPAL

SEQ ID NO: 17 - protein, 8 aa, Homo sapiens
IEHLPGRL

SEQ ID NO: 18 - protein, 6 aa, Homo sapiens
  misc_feature (2): “Xaa” means an unidentified amino acid.
YXLGXG

SEQ ID NO: 19 - protein, 4 aa, Homo sapiens
FPNF

SEQ ID NO: 20 - protein, 8 aa, Homo sapiens
  misc_feature (2): “Xaa” means an unidentified amino acid.
YXLGXGXF

SEQ ID NO: 21 - protein, 7 aa, Homo sapiens
  misc_feature (4)  (5): “Xaa” means an unidentified amino acid.
EVTXXEV

SEQ ID NO: 22 - protein, 8 aa, Homo sapiens
  misc_feature (2): “Xaa” means an unidentified amino acid.
YXLGXGXF

SEQ ID NO: 23 - protein, 11 aa, Homo sapiens
  misc_feature (2): “Xaa” means an unidentified amino acid.
XXVAXXRFPNF

SEQ ID NO: 24 - protein, 8 aa, Mus musculus
LKEGHTSR

SEQ ID NO: 25 - protein, 11 aa, Mus musculus
  misc_feature (4): “Xaa” means an unidentified amino acid.
EHRXTSTWLHR

SEQ ID NO: 26 - protein, 10 aa, Mus musculus
  misc_feature (4): “Xaa” means an unidentified amino acid.
EHRXTSTXLH

SEQ ID NO: 27 - protein, 13 aa, Mus musculus
  misc_feature (1)..(8): “Xaa” means an unidentified amino acid.
XXXXXXXXAVPTK

SEQ ID NO: 28 - protein, 12 aa, Mus musculus
ALVLEELSPTLR

SEQ ID NO: 29 - protein, 7 aa, Mus musculus
IEHLPGR

SEQ ID NO: 30 - protein, 6 aa, Mus musculus
  misc_feature (1): “Xaa” means an unidentified amino acid.
XDGLKT

SEQ ID NO: 31 - protein, 4 aa, Mus musculus
HIIL

SEQ ID NO: 32 - DNA, 492 bp, Homo sapiens
  CDS (1)..(492)
acacctgtctcgcagaccaccacagctgccactgcctcagttagaagcacaaaggacccctgcccctcccagcccccagtgttcccagcagctaagcagtgtccagcattggaagtgacctggccagaggtggaagtgccactgaatggaacgctgagcttatcctgtgtggcctgcagccgcttccccaacttcagcatcctctactggctgggcaatggttccttcattgagcacctcccaggccgactgtgggaggggagcaccagccgggaacgtgggagcacaggtacgcagctgtgcaaggccttggtgctggagcagctgacccctgccctgcacagcaccaacttctcctgtgtgctcgtggaccctgaacaggttgtccagcgtcacgtcgtcctggcccagctctgggctgggctgagggcaaccttgccccccacccaagaagccctgccctccagccacagcagtccacagcagcagggt

SEQ ID NO: 33 - DNA, 495 bp, Mus musculus
  CDS (1)..(495)
acatctgcacctcagacaactgccactgtcttaactggaagctcaaaagacccatgctcttcctggtctccagcagtcccaactaagcagtacccagcactggatgtgatttggccagaaaaagaagtgccactgaatggaactctgaccttgtcctgtactgcctgcagccgcttcccctacttcagcatcctctactggctgggcaatggttccttcattgagcaccttccaggccggctgaaggagggccacacaagtcgcgagcacaggaacacaagcacctggctgcacagggccttggtgctggaagaactgagccccaccctacgaagtaccaacttctcctgtttgtttgtggatcctggacaagtggcccagtatcacatcattctggcccagctctgggatgggttgaagacagctccgtccccttctcaagaaaccctctctagccacagcccagtatccagatcagcaggcccaggggttgca

SEQ ID NO: 34 - DNA, 411 bp, Homo sapiens
  CDS (1)..(411)
acacctgtctcgcagaccaccacagctgccactgcctcagttagaagcacaaaggacccctgcccctcccagcccccagtgttcccagcagctaagcagtgtccagcattggaagtgacctggccagaggtggaagtgccactgaatggaacgctgagcttatcctgtgtggcctgcagccgcttccccaacttcagcatcctctactggctgggcaatggttccttcattgagcacctcccaggccgactgtgggaggggagcaccagccgggaacgtgggagcacaggtacgcagctgtgcaaggccttggtgctggagcagctgacccctgccctgcacagcaccaacttctcctgtgtgctcgtggaccctgaacaggttgtccagcgtcacgtcgtcctggcccag

SEQ ID NO: 35 - DNA, 216 bp, Homo sapiens
  CDS (70)..(216)
tgtgtgactggagaagaggacgttgtcacagataaagagccaggctcaccagctcctgacgcatgcatcatgaccatgagacacaactggacaccagacctcagccctttgtgggtcctgctcctgtgtgcccacgtcgtcactctcctggtcagagccacacctgtctcgcagaccaccacagctgccactgcctcagttagaagcacaaaggac

SEQ ID NO: 36 - DNA, 234 bp, Homo sapiens
  CDS (1)..(141)
ttctcctgtgtgctcgtggaccctgaacaggttgtccagcgtcacgtcgtcctggcccagctctgggctgggctgagggcaaccttgccccccacccaagaagccctgccctccagccacagcagtccacagcagcagggttaagactcagcacagggccagcagcagcacaaccttgaccagagcttgggtcctacctgtctacctggagtgaacagtccctgactgcctgta

SEQ ID NO: 37 - DNA, 744 bp, Homo sapiens
  CDS (160)..(651)
tgtgtgactggagaagaggacgttgtcacagataaagagccaggctcaccagctcctgacgcatgcatcatgaccatgagacacaactggacaccagacctcagccctttgtgggtcctgctcctgtgtgcccacgtcgtcactctcctggtcagagccacacctgtctcgcagaccaccacagctgccactgcctcagttagaagcacaaaggacccctgcccctcccagcccccagtgttcccagcagctaagcagtgtccagcattggaagtgacctggccagaggtggaagtgccactgaatggaacgctgagcttatcctgtgtggcctgcagccgcttccccaacttcagcatcctctactggctgggcaatggttccttcattgagcacctcccaggccgactgtgggaggggagcaccagccgggaacgtgggagcacaggtacgcagctgtgcaaggccttggtgctggagcagctgacccctgccctgcacagcaccaacttctcctgtgtgctcgtggaccctgaacaggttgtccagcgtcacgtcgtcctggcccagctctgggctgggctgagggcaaccttgccccccacccaagaagccctgccctccagccacagcagtccacagcagcagggttaagactcagcacagggccagcagcagcacaaccttgaccagagcttgggtcctacctgtctacctggagtgaacagtccctgactgcctgta

SEQ ID NO: 38 - DNA, 351 bp, Mus musculus
  CDS (1)..(351)
gcagtcccaactaagcagtacccagcactggatgtgatttggccagaaaaagaagtgccactgaatggaactctgaccttgtcctgtactgcctgcagccgcttcccctacttcagcatcctctactggctgggcaatggttccttcattgagcaccttccaggccggctgaaggagggccacacaagtcgcgagcacaggaacacaagcacctggctgcacagggccttggtgctggaagaactgagccccaccctacgaagtaccaacttctcctgtttgtttgtggatcctggacaagtggcccagtatcacatcattctggcccagctctgggatgggttgaagaca

SEQ ID NO: 39 - DNA, 336 bp, Mus musculus
  CDS (151)..(336)
ctgagccttagagctccaagaagctattcggggcttaggagccagaagctgactgctgcctgcccttcccagaaggaggctggcaagctggcaaacggactgttgcttcccagaggaagtcacagacaccagacttgcttgcaagtcatcatgaccatgagacactgctggacagcaggccccagttcttggtgggtcctgcttttgtatgtccatgtcattttggccagagccacatctgcacctcagacaactgccactgtcttaactggaagctcaaaagacccatgctcttcctggtctccagcagtcccaactaagcagtacccagcactg

SEQ ID NO: 40 - DNA, 253 bp, Mus musculus
  CDS (1)..(135)
gatcctggacaagtggcccagtatcacatcattctggcccagctctgggatgggttgaagacagctccgtccccttctcaagaaaccctctctagccacagcccagtatccagatcagcaggcccaggggttgcataaagccaaccacaccatgaccttgaccagagcctggctctcatctacctggagggtggagtctacaccataggctgtgattgcctttctgctgctgaacctcaaactcaagcttcac

SEQ ID NO: 41 - DNA, 847 bp, Mus musculus
  CDS (235)..(729)
ctgagccttagagctccaagaagctattcggggcttaggagccagaagctgactgctgcctgcccttcccagaaggaggctggcaagctggcaaacggactgttgcttcccagaggaagtcacagacaccagacttgcttgcaagtcatcatgaccatgagacactgctggacagcaggccccagttcttggtgggtcctgcttttgtatgtccatgtcattttggccagagccacatctgcacctcagacaactgccactgtcttaactggaagctcaaaagacccatgctcttcctggtctccagcagtcccaactaagcagtacccagcactggatgtgatttggccagaaaaagaagtgccactgaatggaactctgaccttgtcctgtactgcctgcagccgcttcccctacttcagcatcctctactggctgggcaatggttccttcattgagcaccttccaggccggctgaaggagggccacacaagtcgcgagcacaggaacacaagcacctggctgcacagggccttggtgctggaagaactgagccccaccctacgaagtaccaacttctcctgtttgtttgtggatcctggacaagtggcccagtatcacatcattctggcccagctctgggatgggttgaagacagctccgtccccttctcaagaaaccctctctagccacagcccagtatccagatcagcaggcccaggggttgcataaagccaaccacaccatgaccttgaccagagcctggctctcatctacctggagggtggagtctacaccataggctgtgattgcctttctgctgctgaacctcaaactcaagcttcac

SEQ ID NO: 42 - protein, 137 aa, Homo sapiens
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQ

SEQ ID NO: 43 - protein, 49 aa, Homo sapiens
MTMRHNWTPDLSPLWVLLLCAHVVTLLVRATPVSQTTTAATASVRSTKD

SEQ ID NO: 44 - protein, 47 aa, Homo sapiens
FSCVLVDPEQVVQRHVVLAQLWAGLRATLPPTQEALPSSHSSPQQQG

SEQ ID NO: 45 - protein, 164 aa, Homo sapiens; identical to SEQ ID NO: 1

SEQ ID NO: 46 - protein, 117 aa, Mus musculus
AVPTKQYPALDVIWPEKEVPLNGTLTLSCTACSRFPYFSILYWLGNGSFIEHLPGRLKEGHTSREHRNTSTWLHRALVLEELSPTLRSTNFSCLFVDPGQVAQYHIILAQLWDGLKT

SEQ ID NO: 47 - protein, 62 aa, Mus musculus
MTMRHCWTAGPSSWWVLLLYVHVILARATSAPQTTATVLTGSSKDPCSSWSPAVPTKQYPAL

SEQ ID NO: 48 - protein, 45 aa, Mus musculus
DPGQVAQYHIILAQLWDGLKTAPSPSQETLSSHSPVSRSAGPGVA

SEQ ID NO: 49 - protein, 165 aa, Mus musculus; identical to SEQ ID NO: 2

SEQ ID NO: 50 - DNA, 21 bp, Artificial Sequence
  Synthetic
tgtgtgactggagaagaggac

SEQ ID NO: 51 - DNA, 29 bp, Artificial Sequence
  Synthetic
tacaggcagtcagggactgttcactccag

SEQ ID NO: 52 - protein, 14 aa, Artificial Sequence
  Synthetic
ACNCCNGTNWSNCA

SEQ ID NO: 53 - protein, 17 aa, Artificial Sequence
  Synthetic
TGNGCNARNACNACRTG

SEQ ID NO: 54 - protein, 14 aa, Artificial Sequence
  Synthetic
GGRCANGGRTCYTT

SEQ ID NO: 55 - DNA, 21 bp, Artificial Sequence
  Synthetic
ggtcacttccaatgctggaca

SEQ ID NO: 56 - protein, 36 aa, Artificial Sequence
  Synthetic
GGCCACGCGTCGACTAGTACGGGIIGGGIIGGGIIG

SEQ ID NO: 57 - DNA, 21 bp, Artificial Sequence
  Synthetic
gtcctttgtgcttctaactga

SEQ ID NO: 58 - DNA, 35 bp, Artificial Sequence
  Synthetic
gactcgagtcgacatcgattttttttttttttttt

SEQ ID NO: 59 - DNA, 20 bp, Artificial Sequence
  Synthetic
ttctcctgtgtgctcgtgga

SEQ ID NO: 60 - DNA, 17 bp, Artificial Sequence
  Synthetic
gactcgagtcgacatcg

SEQ ID NO: 61 - DNA, 30 bp, Artificial Sequence
  Synthetic
ctcgaggccaccatgaccatgagacacaac

SEQ ID NO: 62 - DNA, 50 bp, Artificial Sequence
  Synthetic
gcggccgctcattagtgatggtgatggtgatgaccctgctgctgtggact

SEQ ID NO: 63 - DNA, 14 bp, Artificial Sequence
  Synthetic
gcngtnccnacnaa

SEQ ID NO: 64 - DNA, 14 bp, Artificial Sequence
  Synthetic
gtyttnarnccrtc

SEQ ID NO: 65 - DNA, 17 bp, Artificial Sequence
  Synthetic
swnctrtgnccytcytt

SEQ ID NO: 66 - DNA, 21 bp, Artificial Sequence
  Synthetic
tgcaggcagtacaggacaagg

SEQ ID NO: 67 - DNA, 21 bp, Artificial Sequence
  Synthetic
gtgctgggtactgcttagttg

SEQ ID NO: 68 - DNA, 18 bp, Artificial Sequence
  Synthetic
gatcctggacaagtggcc

SEQ ID NO: 69 - DNA, 20 bp, Artificial Sequence
  Synthetic
ctgagccttagagctccaag

SEQ ID NO: 70 - DNA, 22 bp, Artificial Sequence
  Synthetic
gtgaagcttgagtttgaggttc

SEQ ID NO: 71 - DNA, 30 bp, Artificial Sequence
  Synthetic
ctcgacgccaccatgaccatgagacactgc

SEQ ID NO: 72 - DNA, 50 bp, Artificial Sequence
  Synthetic
gcggccgctcattagtgatggtgatggtgatgtgcaacccctgggcctgc